(19) United States
(12) Patent Application Publication
(10) Pub. No.: US 2005/0214245 A1
(43) Pub. Date: Sep. 29, 2005

(54) Antibacterial deodorizer kit for refrigerator and purifying system using the same

(75) Inventors: Young Woo Kim, Gimhae City (KR); Sang Ho Park, Changwon City (KR)

Correspondence Address: BIRCH STEWART KOLASCH & BIRCH, PO BOX 747, FALLS CHURCH, VA, 22040-0747, US

(73) Assignee: LG Electronics Inc.

(21) Appl. No.: 11/085,166

(22) Filed: Mar. 22, 2005

(30) Foreign Application Priority Data
Mar. 23, 2004 (KR) 2004-0019808
Mar. 24, 2004 (KR) 2004-0020101

(51) Int. Cl. A61L 9/01

(52) U.S. Cl. 424/76.1

(57) ABSTRACT

In the present invention, a cold air inlet port 32 of a refrigerating chamber 31 of a refrigerator is provided with an antibacterial deodorizer kit 34, which is formed by injection-molding deodorizing agent containing antibacterial capsules 35. The antibacterial capsules 35 are formed by microcapsulizing the antibacterial substance 36. A cold air discharge port 33 of the refrigerating chamber 31 is provided with an anion generating unit 38 for discharging anions into the refrigerating chamber 31.

### BACKGROUND OF THE INVENTION

1. Field of Invention

The present invention relates to a refrigerator. More particularly, the present invention relates to an antibacterial deodorizer kit for a refrigerator that purifies cold air circulating in the refrigerator, and a purifying system using the same.

2. Description of the Prior Art

Hereinafter, a refrigerator according to a prior art will be described with reference to the accompanying drawings.

FIG. 1 shows the internal configuration of the refrigerator according to the prior art.

As shown in the figure, the interior of a refrigerator main body 10 is divided into a freezing chamber 11 and a refrigerating chamber 13. A plurality of shelves 15 are installed in the freezing and refrigerating chambers 11 and 13. The shelves 15 on which foods are placed and stored are detachably installed in a horizontal direction within the freezing and refrigerating chambers 11 and 13.

A vegetable box cover 17 is installed at a lower portion of the refrigerating chamber 13. In addition, a vegetable box 19 is installed in a lower space of the vegetable box cover 17. The vegetable box 19, which is an upwardly opened container, is generally installed to be forwardly drawn in sliding mode. Further, among the shelves 15 installed in the refrigerating chamber, the shelf installed at the lowermost portion functions as the vegetable box cover 17.

In the meantime, the freezing and refrigerating chambers 11 and 13 are selectively opened and closed by a freezing chamber door 21 and a refrigerating chamber door 23 which are pivotably mounted at a side of the main body 10. In addition, a plurality of door baskets 25 for accommodating foods are provided on rear surfaces of the freezing chamber door 21 and the refrigerating chamber door 23.

A plurality of cold air discharge ports 27 are provided in a rear or side surface of the refrigerating chamber 13. The cold air discharge ports 27 are portions through which cold air heat-exchanged in an evaporator (not shown) is discharged into the refrigerating chamber 13. As a plurality of the cold air discharge ports 27 are formed in a portion where the cold air can be directly supplied into the refrigerating chamber, for example, the rear surface of the refrigerating chamber, the cold air discharge ports 27 may be formed in a portion where the cold air can also be directly supplied into the vegetable box 19, for example, the direct rear surface of the vegetable box.

However, the related art refrigerator so configured has the following problems.

In the prior art, the cold air discharge ports 27 are provided with a deodorizing agent (not shown) for removing smell from the cold air circulating in the refrigerating chamber 13. However, since the deodorizing agent performs only a deodorizing function, it cannot remove harmful substances such as a variety of bacteria from the cold air circulating in the refrigerating chamber 13.

When foods such as vegetables and fruits are accommodated in an accommodation space of the vegetable box 19, it is possible to keep the vegetables and fruits fresh. However, when they are kept in the vegetable box 19 for a long time, there is a disadvantage in that active oxygen generated in the refrigerating chamber 13 or the vegetable box has a bad effect on the foods.

### SUMMARY OF THE INVENTION

The present invention is conceived to solve the aforementioned problems in the prior art. Accordingly, an object of the present invention is to provide an antibacterial deodorizer kit for a refrigerator adapted to remove various harmful substances from cold air circulating in a storage space of the refrigerator, and a purifying system using the same.

Another object of the present invention is to provide a food antioxidant kit for a refrigerator which can prevent foods stored in a refrigerating chamber including a vegetable chamber from oxidizing.

According to an aspect of the present invention for achieving the objects, there is provided an antibacterial deodorizer kit for a refrigerator, comprising an antibacterial agent for removing bacteria from cold air circulating in a storage space of the refrigerator; and a deodorizing agent for removing smell from the cold air. The antibacterial agent comprises a plurality of antibacterial capsules where an antibacterial substance is microcapsulized, and is integrally formed with the deodorizing agent.

Preferably, the antibacterial substance of the antibacterial agent is one selected from a group consisting of rosemary, flavonoid and catechin that are polyphenols, the deodorizing agent is a substance selected from a group consisting of activated carbon, zeolite, and ceramic, and the antibacterial deodorizer kit is injection-molded.

A ratio of the antibacterial agent to be contained in the antibacterial deodorizer kit may be within a range of 0.1% to 30%, preferably 1% or more.

A purifying system for a refrigerator of the present invention comprises an antibacterial deodorizer kit which includes a microcapsulized antibacterial agent for removing bacteria from cold air circulating in the refrigerator and a deodorizing agent for removing smell from the cold air; and an anion generating unit for discharging anions to the interior of the refrigerator. The antibacterial deodorizer kit and the anion generating unit are installed at a cold air inlet port and a cold air discharge port, through which the cold air is ejected from and discharged into a storage space of the refrigerator, respectively.

A food antioxidant kit for a refrigerator of the present invention comprises a substrate installed in a storage space of the refrigerator; and antioxidant capsules provided in the substrate and formed by microcapsulizing an antioxidant substance to prevent nutrients contained in foods stored in the storage space from oxidizing by means of active oxygen.

The antioxidant substance may include polyphenols.

The food antioxidant kit may further comprise a case for installing the substrate in the refrigerator.

The substrate may be either nonwoven fabric in which the antioxidant capsules are provided in the form of particles or injection-molded plastic with the antioxidant capsules contained therein.

According to another aspect of the present invention, there is provided a purifying system for a refrigerator, comprising a food antioxidant kit comprising a substrate installed in a storage space of the refrigerator, and antioxidant capsules provided in the substrate and formed by microcapsulizing antioxidant substance to prevent nutrients contained in foods stored in the storage space from oxidizing by means of active oxygen; and an anion generating unit for discharging anions to an interior of the refrigerator. The food antioxidant kit and the anion generating unit are installed at a cold air inlet port and a cold air discharge port, through which the cold air is ejected from and discharged into a storage space of the refrigerator, respectively.

According to the present invention so configured, it is expected that it is possible to keep the stored foods fresh and to prevent the foods from being oxidized since various bacteria and smell contained in the cold air circulating in the storage space of the refrigerator are removed.

### BRIEF DESCRIPTION OF THE DRAWINGS

The above and other objects, features and advantages of the present invention will become apparent from the following description of preferred embodiments given in conjunction with the accompanying drawings, in which:

FIG. 1 is a perspective view showing an interior of a refrigerator according to a prior art.

FIG. 2 is a schematic view showing a storage space of a refrigerator which is provided with a preferred embodiment of a purifying system according to the present invention.

FIG. 3 is an enlarged view showing antibacterial substance of the embodiment shown in FIG. 2.

FIG. 4 is an exemplary view of a vegetable box which is provided with a food antioxidant kit according to another embodiment of the present invention.

FIG. 5 is an exemplary view of an antioxidant capsule according to the other embodiment of the present invention.

FIG. 6 is an exemplary view showing an operation of the other embodiment of the present invention.

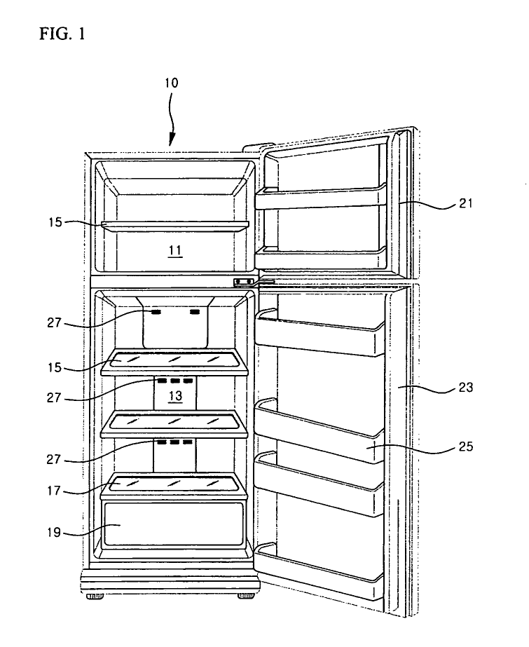

### DETAILED DESCRIPTION OF THE PREFERRED EMBODIMENT

Hereinafter, preferred embodiments of an antibacterial deodorizer kit for a refrigerator and a purifying system using the same according to the present invention will be described in detail with reference to the accompanying drawings.

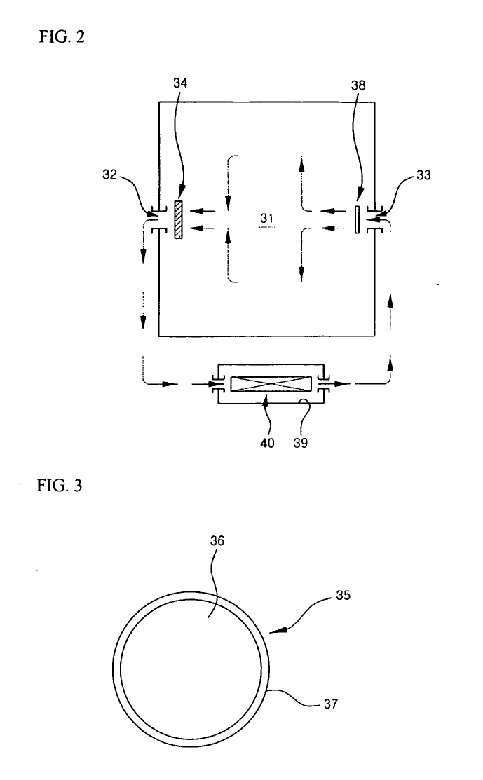

FIG. 2 schematically shows a storage space of a refrigerator in which the purifying system according to the present invention and flow of air therein; and FIG. 3 illustrates antibacterial substance for purifying the air in the refrigerator.

As shown in the figures, a cold air inlet port 32 and a cold air discharge port 33 are provided at both sides of a refrigerating chamber 31 of the refrigerator, respectively. The cold air inlet port 32 functions as a passage through which relatively high-temperature air circulated in the refrigerating chamber 31 is ejected from the refrigerating chamber 31 such that the air is heat-exchanged in a heat exchanger 40 to be described later. The cold air discharge port 33 functions as an outlet through which the air heat-exchanged in the heat exchanger 40 is discharged into the refrigerating chamber 31.

Furthermore, the cold air inlet port 32 is equipped with an antibacterial deodorizer kit 34. The antibacterial deodorizer kit 34 includes antibacterial agent for removing various bacteria from the cold air which passes the cold air inlet port 32 after circulating in the refrigerating chamber 31, and deodorizing agent for removing smell contained therein.

As the deodorizing agent of the antibacterial deodorizer kit 34, the substance with deodorizing function such as ceramic, zeolite, or activated carbon may be used. The antibacterial substance 36 is made of polyphenols. The polyphenols are contained in catechin of green tea or herbs such as rosemary or flavonoid.

The antibacterial deodorizer kit 34 is formed by injection-molding the activated carbon containing antibacterial agent and is installed in the refrigerating chamber while being provided in an additional case, container or the like (not shown). Alternatively, the antibacterial deodorizer kit 34 may be installed in the front of the cold air inlet port 32 as illustrated in the embodiment.

At this time, according to a ratio of the antibacterial agent to be contained in the antibacterial deodorizer kit 34, there is a tradeoff between antibacterial and deodorizing effects. That is, when the ratio of the antibacterial agent in the antibacterial deodorizer kit 34 increases, the antibacterial effect increases while the deodorizing effect decreases. Therefore, in order to secure predetermined antibacterial and deodorizing effects, the ratio of the antibacterial agent in the antibacterial deodorizer kit 34 may be within about 0.01 to 30%, preferably 1% or more.

In the meantime, as shown in FIG. 3, it is preferred that the antibacterial agent of the antibacterial deodorizer kit 34 be enclosed with a plurality of capsules 35 where the antibacterial substance 36 in liquid or solid phase is microcapsulized. The antibacterial deodorizer kit 34 having a predetermined shape is completed by molding (for example, injection-molding) the antibacterial agent capsules together with the aforementioned deodorizing agent.

A microcapsule 37 for microcapsulizing the antibacterial substance 36 prevents the antibacterial substance 36 from being destroyed by high temperature in the injection-molding process of the antibacterial deodorizer kit 34, and simultaneously serves to cause the antibacterial substance 36 to gradually radiate into the refrigerating chamber. In addition, the micro capsule 37 may be made of polyvinylalcohol, polypropylene or the like.

The cold air discharge port 33 is provided with an anion generating unit 38. The anion generating unit 38 serves to radiate anions into the cold air discharged into the refrigerating chamber 31 through the cold air discharge port 33. The anion generating unit 38 may be formed of plastic which is coated with anion or injection-molded with anion contained. Amounts of anions generated from the anion generating unit 38 may be determined by external stimulus such as heating, supply of moisture, and friction with fluid.

In the meantime, there is provided a heat exchange chamber 39 in communication with the cold air inlet port 32 and the cold air discharge port 33. The heat exchange chamber 39 is formed with a predetermined space for heat exchange, in which an evaporator 40 is installed. The evaporator 40 serves to heat-exchange the cold air sucked therein through the cold air inlet port 32 with refrigerant flowing therein. In addition, a blowing fan (not shown) for discharging the cold air through the cold air discharge port 33 is installed in the heat exchange chamber 39.

Next, a purifying process of cold air according to the preferred embodiment of the purifying system for a refrigerator of the present invention so configured will be described below.

First, when the blowing fan installed in the heat exchange chamber 39 operates, the cold air is supplied into the refrigerating chamber in the refrigerator through the cold air discharge port 33. The cold air has had a lower temperature by the evaporator installed in the heat exchange chamber 39, and then the cold air supplied into the refrigerating chamber cools foods accommodated therein while circulating therein.

The cold air discharged through the cold air discharge port 33 passes through the anion generating unit 38. At this time, the anion generating unit 38 stimulated by the flow of the cold air generates anions, and the anions are discharged along with the cold air into the refrigerating chamber 31. The anions also alkalify the foods accommodated in the refrigerating chamber 31 and prevent them from aging, and thus serve to keep them fresh.

Further, the cold air supplied into the refrigerating chamber is heated to a relatively higher temperature due to the heat exchange with the foods accommodated therein. The air with the relatively higher temperature is again returned to the heat exchange chamber 39 through the cold air inlet port 32.

Before the air is sucked into the heat exchange chamber 39 through the cold air inlet port 32, the air passes through the antibacterial deodorizer kit 34. At this time, various bacteria contained in the cold air are removed by the antibacterial deodorizer kit 34, i.e., the antibacterial substance 36 of the antibacterial capsules 35. The smell contained in the cold air is also removed by means of the deodorizing agent of the antibacterial deodorizer kit 34.

While the cold air sucked in the heat exchange chamber 39 through the cold air inlet port 32 passes through the evaporator 40 in the heat exchange chamber 39, the cold air is heat-exchanged with the refrigerant flowing in the interior of the evaporator 40. Then, when the blowing fan continuously operates, the aforementioned circulation of the air discharged into the refrigerating chamber 31 through the cold air discharge port 33 is continuously performed.

Next, another embodiment of the present invention will be described.

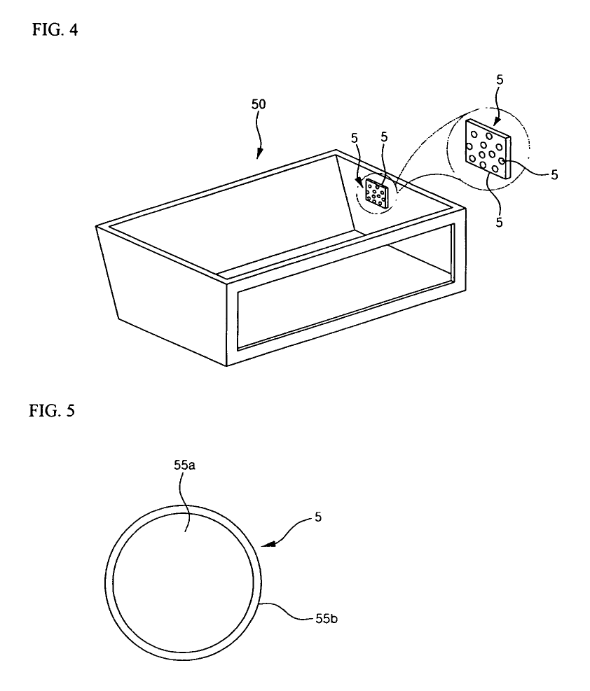

FIG. 4 shows a vegetable box which is provided with a food antioxidant kit for a refrigerator according to the other embodiment of the present invention; and FIG. 5 shows a particle of antioxidant substance.

As shown in the figures, a predetermined accommodation space is defined in a vegetable box 50 installed in the refrigerating chamber. The vegetable box 50 is generally shaped in a hexahedron with an upper face opened. Foods such as vegetables and fruits can be accommodated and stored in the vegetable box 50 to be fresh. As described above, a portion of the cold air supplied into refrigerating chamber is directly guided into the vegetable box 50, so that the vegetables and fruits therein are generally stored to be fresher. In addition, according to the present embodiment, a food antioxidant kit 53 is installed on a side of the interior of the vegetable box.

The food antioxidant kit 53 comprises a substrate 54 fixed on the side of the interior of the vegetable box 50 and antioxidant capsules 55 provided in the substrate 54. As the substrate 54, nonwoven fabric with the antioxidant capsules 55 provided therein or plastic injection-molded with the antioxidant capsules 55 contained therein may be used. Particularly, when the substrate 54 is the nonwoven fabric, an additional case (not shown) for fixing the substrate 54 to the accommodation space 31′ is needed.

The antioxidant capsules 55 serve to prevent the foods accommodated in the vegetable box 50 from being oxidized by active oxygen in the refrigerating chamber. The antioxidant capsules 55 are formed by causing antioxidant substance 55a to be capsulized by micro capsules 55b, as shown in FIG. 5.

The antioxidant substance 55a, instead of nutrients contained in the foods accommodated in the vegetable box 50, combines with the active oxygen in the refrigerating chamber and then serves to be oxidized. As the antioxidant substance 55a, polyphenols are used. The polyphenols are contained in green tea or herbs such as rosemary or flavonoid.

The micro capsules 55b for microcapsulizing the antioxidant substance 55a serve to control the rate at which the antioxidant substance 55a is discharged into the interior of the vegetable box and to protect the antioxidant substance when the antioxidant substance 55a is introduced into the substrate 54. As the micro capsules 55b, polyvinylalcohol, polypropylene or the like may be used. The antioxidant capsule 55 containing the micro capsule 55b is 1 micrometer or more in diameter, preferably about 10 micrometers.

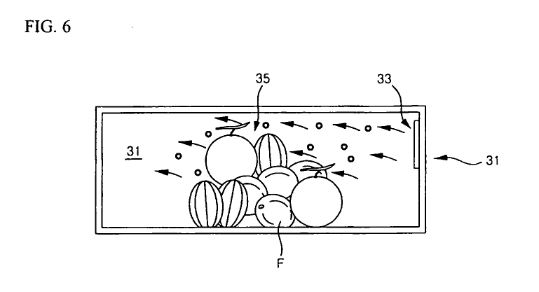

Next, a process of preventing foods from being oxidized by the preferred embodiment of the food antioxidant kit for a refrigerator according to the present invention so configured will be described with reference to FIG. 6.

As shown in the figure, foods F such as vegetables and fruits are accommodated and stored in the accommodation space of the vegetable box 50 for a refrigerator. In such a state where the foods F are accommodated, the antioxidant substance 55a is discharged from the food antioxidant kit 53 installed on the side of the interior of the vegetable box 50.

However, since the discharging rate of the antioxidant substance 55a is controlled by the micro capsules 55b, the antioxidant substance 55a is discharged not at a time but for a long time at a constant rate from the food antioxidant kit 53 into the interior of the vegetable box. In addition, the discharging rate of the antioxidant substance 55a may be changed according to external stimulus, an amount of moisture, or the like.

In the meantime, the antioxidant substance 55a discharged into the interior of the vegetable box 50 is combined with active oxygen in the vegetable box 50 instead of the foods F. Since the antioxidant substance 55a is oxidized by the active oxygen, the oxidization of the foods F can be suppressed to the utmost. Thus, since nutrients contained in the foods F are not destroyed, the foods F are not aged and thus can be stored freshly without withering.

It is noted that instead of the antibacterial deodorizing agent of the first embodiment, the antioxidant substance is microcapsulized and used in the present embodiment. Furthermore, although in the present embodiment, the vegetable box is described as an example of the storage space for keeping the foods fresh, the food antioxidant kit 53 may be attached to the interior of the refrigerating chamber as described in the first embodiment.

Furthermore, instead of the antibacterial deodorizer kit of the first embodiment, the antioxidant kit may be installed to the interior of the refrigerating chamber in the present embodiment.

According to the antibacterial deodorizer kit for a refrigerator of the present invention and the purifying system using the same so constructed, the following advantages can be expected.

First, it is possible to simply remove various bacteria and smell from cold air circulating in the storage space of the refrigerator. Since anions are radiated into the cold air circulating in the storage space, it is also to keep foods stored in the storage space fresh. It is noted that the kit using the capsulized antioxidant substance instead of antibacterial deodorizing agent suppresses to the utmost the foods from being oxidized by the air in the refrigerator, thus keeping the foods fresh.

The scope of the present invention is not limited to the embodiments described and illustrated above but is defined by the appended claims. It will be apparent that those skilled in the art can make various modifications and changes thereto within the scope of the technical spirit of the invention. Therefore, the true scope of the present invention should be defined by the appended claims.

1. An antibacterial deodorizer kit for a refrigerator, comprising:
an antibacterial agent for removing bacteria from cold air circulating in a storage space of the refrigerator; and
a deodorizing agent for removing smell from the cold air,
wherein the antibacterial agent comprises a plurality of antibacterial capsules where an antibacterial substance is microcapsulized, and is integrally formed with the deodorizing agent.

2. The antibacterial deodorizer kit as claimed in claim 1, wherein the antibacterial substance of the antibacterial agent is one selected from a group consisting of rosemary, flavonoid and catechin that are polyphenols, the deodorizing agent is a substance selected from a group consisting of activated carbon, zeolite, and ceramic, and the antibacterial deodorizer kit is injection-molded.

3. The antibacterial deodorizer kit as claimed in claim 1 or 2, wherein a ratio of the antibacterial agent to be contained in the antibacterial deodorizer kit is within a range of 0.1% to 30%.

4. The antibacterial deodorizer kit as claimed in claim 3, wherein the ratio of the antibacterial agent to be contained in the antibacterial deodorizer kit is 1% or more.

5. A purifying system for a refrigerator, comprising:
an antibacterial deodorizer kit including a microcapsulized antibacterial agent for removing bacteria from cold air circulating in the refrigerator and a deodorizing agent for removing smell from the cold air; and
an anion generating unit for discharging anions to the interior of the refrigerator,
wherein the antibacterial deodorizer kit and the anion generating unit are installed at a cold air inlet port and a cold air discharge port, through which the cold air is ejected from and discharged into a storage space of the refrigerator, respectively.

6. The purifying system as claimed in claim 5, wherein the antibacterial substance of the antibacterial agent is one selected from a group consisting of rosemary, flavonoid and catechin that are polyphenols, the deodorizing agent is a substance selected from a group consisting of activated carbon, zeolite, and ceramic, and the antibacterial deodorizer kit is injection-molded.

7. The purifying system as claimed in claim 5, wherein a ratio of the antibacterial agent to be contained in the antibacterial deodorizer kit is within a range of 0.1% to 30%.

8. The purifying system as claimed in claim 7, wherein the ratio of the antibacterial agent to be contained in the antibacterial deodorizer kit is 1% or more.

9. The purifying system as claimed in claim 5, wherein the antibacterial agent is contained in and integrally formed with the deodorizing agent.

10. A food antioxidant kit for a refrigerator, comprising:
a substrate installed in a storage space of the refrigerator; and
antioxidant capsules provided in the substrate and formed by microcapsulizing an antioxidant substance to prevent nutrients contained in foods stored in the storage space from oxidizing by means of active oxygen.

11. The food antioxidant kit as claimed in claim 10, wherein the antioxidant substance is a class of polyphenols.

12. The food antioxidant kit as claimed in claim 10, further comprising a case used to install the substrate within the refrigerator.

13. The food antioxidant kit as claimed in claim 10, wherein the substrate is nonwoven fabric in which the antioxidant capsules are provided in the form of particles.

14. The food antioxidant kit as claimed in claim 10, wherein the substrate is injection-molded plastic with the antioxidant capsules contained therein.

15. A purifying system for a refrigerator, comprising:
a food antioxidant kit comprising a substrate installed in a storage space of the refrigerator, and an antioxidant capsule provided in the substrate and formed by microcapsulizing an antioxidant substance to prevent nutrients contained in foods stored in the storage space from oxidizing by means of active oxygen; and
an anion generating unit for discharging anions to the interior of the refrigerator,
wherein the food antioxidant kit and the anion generating unit are installed at a cold air inlet port and a cold air discharge port, through which the cold air is ejected from and discharged into a storage space of the refrigerator, respectively.
